(19) United States
(12) Patent Application Publication
(10) Pub. No.: US 2015/0217094 A1
(43) Pub. Date: Aug. 6, 2015

(54) Fill Valve

(71) Applicant: Applied Medical Technology, Inc., Brecksville, OH (US)

(72) Inventors: Grant Wesley Phillips, Richfield, OH (US); Derek M. Williams, Cuyahoga Falls, OH (US); George J. Picha, Brecksville, OH (US)

(73) Assignee: Applied Medical Technology, Inc., Brecksville, OH (US)

(21) Appl. No.: 14/286,319

(22) Filed: May 23, 2014

Related U.S. Application Data
(60) Provisional application No. 61/935,940, filed on Feb. 5, 2014.

(51) Int. Cl. A61M 25/10; A61J 15/00

(52) U.S. Cl. CPC A61M 25/10185; A61J 15/0049

(57) ABSTRACT

A fill valve for mediating two-way fluid communication between a medical instrument and a medical device is provided. The fill valve comprises a valve element and a valve housing. The valve element comprises a valve element body extending along a major axis, a valve element first end comprising a recessed internal surface configured to provide a sealing contact engagement with a medical instrument, a valve element second end, and a valve element body external surface. The valve element is resilient and deformable. The valve element also has a hole that extends along the major axis and that is substantially cylindrically symmetrical with respect to the major axis. The valve housing is configured to hold the valve element in place by a circumferential compression fit. An enteral feeding button comprising such a fill valve, and a valve element of such a fill valve, are also provided.

### CROSS-REFERENCE TO RELATED APPLICATIONS

This application claims the benefit of U.S. Provisional Application No. 61/935,940, filed Feb. 5, 2014, the entire disclosure of which is hereby incorporated herein by reference.

### TECHNICAL FIELD

The present invention relates generally to a fill valve, and more particularly to a fill valve for mediating two-way fluid communication between a medical instrument and a medical device, an enteral feeding button comprising such a fill valve, and a valve element of such a fill valve.

### BACKGROUND

Fill valves can be important components of medical devices. For example, fill valves can be used with low-profile enteral feeding devices, such as enteral feeding buttons, to mediate inflation and deflation of balloons coupled to the low-profile enteral feeding devices. Examples of such fill valves include check valves, syringe-access check valves, and Luer-access check valves. A medical instrument, such as a syringe, can be used to inflate or deflate the balloon through the fill valve.

Many fill valves consist of three parts: a rigid plastic housing, an elastomeric gasket, and a spring. Such fill valves can be operated as follows. A syringe tip enters the housing and pushes down on the elastomeric gasket, which causes the spring to compress. In this “open” state of the valve, the syringe can now push or pull a fluid, e.g. gas or liquid, through the valve. Once the syringe tip is removed from the housing, the spring pushes on the gasket, which creates a tight seal against the rigid housing of the fill valve, thus creating the normally “closed” state of the valve. An example of this type of valve is the Halkey-Roberts Low-Profile Check Valve.

Other fill valves use the elastomeric properties of the gasket to function like a spring, thus eliminating the need for a spring and allowing for a fill valve consisting of only two components. Elimination of the spring is advantageous because springs are often made of metal, and metals, including non-ferrous ones, e.g. certain stainless steels and titanium, among others, can distort magnetic resonance images when MRI studies are carried out on patients for which such valves are in use. However, due to the gasket having to act as a gasket and like a spring, these two-part fill valves are usually larger than the traditional three-part fill valves described above.

One example of this type of two part valve is the Halkey-Roberts Swabable Valve. The valve is made of a silicone gasket shaped like a cone with a slit through the top. When a male Luer is inserted into the valve, the tip of the male Luer compresses the silicone gasket, causing the slit to open up and allowing fluid to flow through the valve. When the male Luer is retracted, the compression force on the silicone gasket is removed and the silicone gasket acts like a spring and returns to its original position. The housing around the silicone gasket causes the slit to close tightly and seals up to 30 psi back pressure.

Another two part valve is the Halkey-Roberts Luer Activated Check Valve. This is a two part valve that also does not use a spring. The elastomeric properties of the gasket also function like a spring, but the valve is very long, approximately three times longer than the Halkey-Roberts Low Profile Valve.

There are also two-part septum valves that can be used as a fill valve/check valve. These valves consist of a rigid housing and a type of self-sealing membrane. Only sharp needles or specialized blunt needles can be used in conjunction with these septum valves. Due to the sharp or specialized blunt needles involved, usually only trained healthcare professionals use and access these septum valves.

Many of the problems associated with use of these and other fill valves with low-profile enteral feeding devices apply to use of fill valves with other medical devices too, e.g. cecostomy buttons, intravenous lines, arterial lines, dialysis lines, Foley catheters, and chest tubes, among other medical devices.

Accordingly, there is a need for fill valves for use with low-profile enteral feeding devices, such as enteral feeding buttons, as well as for use with medical devices generally, that address these issues and provide improvements.

### SUMMARY

The fill valve for mediating two-way fluid communication between a medical instrument and a medical device, disclosed herein, is such a fill valve.

In a first aspect of the disclosure, a fill valve for mediating two-way fluid communication between a medical instrument and a medical device is provided. The fill valve comprises a valve element. The valve element comprises (i) a valve element body extending along a major axis of the valve element, (ii) a valve element first end comprising a recessed internal surface configured to provide a sealing contact engagement with a medical instrument, (iii) a valve element second end opposite the valve element first end, and (iv) a valve element body external surface. The valve element is resilient and deformable. The valve element also has a hole that extends along the major axis, from the valve element first end, to the valve element second end, through the valve element body, and that is substantially cylindrically symmetrical with respect to the major axis. The fill valve also comprises a valve housing. The valve housing is configured to hold the valve element in place by a circumferential compression fit with the valve element body external surface. The valve housing also is configured to connect to, or be an integral part of, a medical device. The fill valve is configured to, in the absence of the sealing contact engagement, maintain the hole in a closed configuration, from the valve element first end, to the valve element second end, through the valve element body, based on the circumferential compression fit, thereby preventing two-way fluid communication through the hole. The fill valve also is configured to, in the presence of the sealing contact engagement, deform the hole, reversibly, to an open configuration, from the valve element first end, to the valve element second end, through the valve element body, despite the circumferential compression fit, thereby allowing two-way fluid communication through the hole.

In an example of the first aspect, the valve element is made of an elastomeric material selected from the group consisting of silicone, polyurethane, styrene ethylene butylene styrene block copolymer, polyvinylchloride, and combinations thereof.

In another example of the first aspect, the valve housing is made of an elastomeric material selected from the group consisting of silicone, polyurethane, styrene ethylene butylene styrene block copolymer, polyvinylchloride, and combinations thereof.

In another example of the first aspect, the valve element is substantially cylindrical.

In another example of the first aspect, (i) the valve element has a valve element external diameter, (ii) the valve housing has a valve housing internal diameter, and (iii) the valve element external diameter is greater than the valve housing internal diameter.

In another example of the first aspect, the hole is centered with respect to the valve element.

In another example of the first aspect, the valve element is centered with respect to the valve housing.

In another example of the first aspect, the hole deforms to a truncated conical shaped opening in the open configuration.

In another example of the first aspect, the recessed internal surface defines a concave depression receding along the major axis, and the hole extends from the concave depression, to the valve element second end, through the valve element body.

In another example of the first aspect, the valve element has a valve element length along the major axis, the hole has a hole length along the major axis, and the hole length is less than the valve element length.

In another example of the first aspect, the valve housing includes an inner gripping portion.

In another example of the first aspect, the medical instrument with which the recessed internal surface of the valve element first end is configured to provide the sealing contact engagement is a syringe, a cannula, or a tube.

In another example of the first aspect, the medical device to which the valve housing is configured to be connected to, or to be an integral part of, is an enteral feeding device, an enteral feeding button, a gastrostomy balloon button, a gastrostomy-jejunostomy balloon button, a jejunostomy balloon button, a cecostomy balloon button, a balloon gastrostomy tube, an intravenous line, an arterial line, a dialysis line, a Foley catheter, or a chest tube.

In a second aspect of the disclosure, an enteral feeding button is provided. The enteral feeding button comprises an enteral feeding button body, an enteral feeding tube, and a balloon. The enteral feeding button body, the enteral feeding tube, and the balloon are connected in series in fluid communication. The enteral feeding button body comprises a channel for inflating or deflating the balloon, a channel for receiving a feeding solution, and a fill valve for mediating two-way fluid communication between a medical instrument and the channel for inflating or deflating the balloon. The fill valve comprises a valve element. The valve element comprises (i) a valve element body extending along a major axis of the valve element, (ii) a valve element first end comprising a recessed internal surface configured to provide a sealing contact engagement with the medical instrument, (iii) a valve element second end opposite the valve element first end, and (iv) a valve element body external surface, wherein the valve element is resilient and deformable and has a hole that extends along the major axis, from the valve element first end, to the valve element second end, through the valve element body, and that is substantially cylindrically symmetrical with respect to the major axis. The fill valve also comprises a valve housing. The valve housing is configured to hold the valve element in place by a circumferential compression fit with the valve element body external surface. The valve housing is an integral part of the enteral feeding button body. The fill valve is configured to, in the absence of the sealing contact engagement, maintain the hole in a closed configuration, from the valve element first end, to the valve element second end, through the valve element body, based on the circumferential compression fit, thereby preventing two-way fluid communication through the hole. The fill valve also is configured to, in the presence of the sealing contact engagement, deform the hole, reversibly, to an open configuration, from the valve element first end, to the valve element second end, through the valve element body, despite the circumferential compression fit, thereby allowing two-way fluid communication through the hole.

In an example of the second aspect, the valve element is made of an elastomeric material selected from the group consisting of silicone, polyurethane, styrene ethylene butylene styrene block copolymer, polyvinylchloride, and combinations thereof.

In another example of the second aspect, the valve housing is made of an elastomeric material selected from the group consisting of silicone, polyurethane, styrene ethylene butylene styrene block copolymer, polyvinylchloride, and combinations thereof.

In another example of the second aspect, the valve element is substantially cylindrical.

In another example of the second aspect, (i) the valve element has a valve element external diameter, (ii) the valve housing has a valve housing internal diameter, and (iii) the valve element external diameter is greater than the valve housing internal diameter.

In another example of the second aspect, the hole is centered with respect to the valve element.

In another example of the second aspect, the valve element is centered with respect to the valve housing.

In another example of the second aspect, the hole deforms to a truncated conical shaped opening in the open configuration.

In another example of the second aspect, the recessed internal surface defines a concave depression receding along the major axis, and the hole extends from the concave depression, to the valve element second end, through the valve element body.

In another example of the second aspect, the valve element has a valve element length along the major axis, the hole has a hole length along the major axis, and the hole length is less than the valve element length.

In another example of the second aspect, the valve housing includes an inner gripping portion.

In another example of the second aspect, the medical instrument with which the recessed internal surface of the valve element first end is configured to provide the sealing contact engagement is a syringe, a cannula, or a tube.

In a third aspect of the disclosure, a valve element is provided. The valve element comprises (i) a valve element body extending along a major axis of the valve element, (ii) a valve element first end comprising a recessed internal surface configured to provide a sealing contact engagement with a medical instrument, (iii) a valve element second end opposite the valve element first end, and (iv) a valve element body external surface. The valve element is resilient and deformable. The valve element also has a hole that extends along the major axis, from the valve element first end, to the valve element second end, through the valve element body, and that is substantially cylindrically symmetrical with respect to the major axis. The valve element is configured to, in the absence of the sealing contact engagement, maintain the hole in a closed configuration, from the valve element first end, to the valve element second end, through the valve element body, based on a circumferential compression fit between a valve housing and the valve element body external surface, thereby preventing two-way fluid communication through the hole. The valve element also is configured to, in the presence of the sealing contact engagement, deform the hole, reversibly, to an open configuration, from the valve element first end, to the valve element second end, through the valve element body, despite the circumferential compression fit, thereby allowing two-way fluid communication through the hole.

In an example of the third aspect, the valve element is made of an elastomeric material selected from the group consisting of silicone, polyurethane, styrene ethylene butylene styrene block copolymer, polyvinylchloride, and combinations thereof.

In another example of the third aspect, the valve element is substantially cylindrical.

In another example of the third aspect, the hole is centered with respect to the valve element.

In another example of the third aspect, the hole deforms to a truncated conical shaped opening in the open configuration.

In another example of the third aspect, the recessed internal surface defines a concave depression receding along the major axis, and the hole extends from the concave depression, to the valve element second end, through the valve element body.

In another example of the third aspect, the valve element has a valve element length along the major axis, the hole has a hole length along the major axis, and the hole length is less than the valve element length.

In another example of the third aspect, the medical instrument with which the recessed internal surface of the valve element first end is configured to provide the sealing contact engagement is a syringe, a cannula, or a tube.

### BRIEF DESCRIPTION OF THE DRAWINGS

These and other features, aspects, and advantages of the present disclosure are better understood when the following detailed description is read with reference to the accompanying drawings, in which:

FIG. 1 is a schematic perspective view of a fill valve for mediating two-way fluid communication between a medical instrument, in this case a syringe, and a medical device, in this case a gastrostomy balloon button, in which the valve housing of the fill valve is an integral part of the medical device and in which the balloon of the gastrostomy balloon button is not shown;

FIG. 2 is a schematic sectional side view of the fill valve of FIG. 1;

FIG. 3 is a schematic sectional perspective view of the fill valve of FIG. 1;

FIG. 4 is a schematic perspective view of a fill valve for mediating two-way fluid communication between a medical instrument, in this case a syringe, and a medical device, in this case a gastrostomy balloon button, in which the valve housing of the fill valve is an integral part of the medical device and in which the balloon of the gastrostomy balloon button is not shown, and further in which the tip of the syringe is inserted in the valve element of the fill valve;

FIG. 5 is a schematic sectional side view of the fill valve of FIG. 4, in which the tip of the syringe is inserted in the valve element of the fill valve;

FIG. 6 is a schematic sectional perspective view of the fill valve of FIG. 4, in which the tip of the syringe is inserted in the valve element of the fill valve;

FIG. 7 is a schematic top view of the valve element first end of the valve element of the fill valve of FIG. 1;

FIG. 8 is a schematic sectional side view, taken along line 8-8 of FIG. 7, of the valve element of the fill valve of FIG. 1;

FIG. 9 is a schematic sectional perspective view, taken along line 8-8 of FIG. 7, of the valve element of the fill valve of FIG. 1;

FIG. 10 is a schematic top view of the valve element first end of the valve element of the fill valve of FIG. 4, in which the tip of the syringe is inserted in the valve element;

FIG. 11 is a schematic sectional side view, taken along line 11-11 of FIG. 10, of the valve element of the fill valve of FIG. 4, in which the tip of the syringe is inserted in the valve element;

FIG. 12 is a schematic sectional perspective view, taken along line 11-11 of FIG. 10, of the valve element of the fill valve of FIG. 4, in which the tip of the syringe is inserted in the valve element; and

FIG. 13 is a schematic perspective view of an enteral feeding button including an enteral feeding button body, an enteral feeding tube, and a balloon, in which the enteral feeding button is a gastrostomy balloon button, the enteral feeding button body includes a fill valve including a valve element and a valve housing, and the valve housing is an integral part of the enteral feeding button body, and in which the balloon is deflated.

FIG. 14 is a schematic sectional view of the enteral feeding button of FIG. 13.

FIG. 15 is a schematic perspective view of an enteral feeding button including an enteral feeding button body, an enteral feeding tube, and a balloon, in which the enteral feeding button is a gastrostomy balloon button, the enteral feeding button body includes a fill valve including a valve element and a valve housing, and the valve housing is an integral part of the enteral feeding button body, and in which the balloon is inflated.

FIG. 16 is a schematic sectional view of the enteral feeding button of FIG. 15, in which a tip of a syringe is inserted in the valve element of the fill valve.

### DETAILED DESCRIPTION

The subject matter of the present disclosure will now be described more fully hereinafter with reference to the accompanying drawings in which example embodiments are shown. Whenever possible, the same reference numerals are used throughout the drawings to refer to the same or like parts. However, this subject matter may be embodied in many different forms and should not be construed as limited to the embodiments set forth herein. These example embodiments are provided so that this disclosure will be both thorough and complete, and will fully convey the scope of the claims that follow to those skilled in the art.

As set forth in the figures, an example fill valve for mediating two-way fluid communication between a medical instrument and a medical device is provided. The fill valve can provide numerous advantages. For example, the fill valve can be small and have a low profile, thus allowing a low profile also for a medical device, e.g. a low-profile enteral feeding device, such as an enteral feeding button, to which the fill valve is connected, or of which the fill valve is an integral part. The fill valve can include a valve element that is soft and that thus can be activated by a variety of readily available types of syringes, including a Luer-slip type syringe, a Luer-lock type syringe, an oral-tip type syringe, and an enteral-tip type syringe, among others, thereby providing a high degree of compatibility between the fill valve and a variety of types of syringes. The fill valve can include a valve housing that is soft, thus avoiding use of hard and/or rigid valve housings and patient discomfort associated therewith. The fill valve can be sufficiently free of metals so as to avoid distorting or affecting magnetic resonance imaging studies. The fill valve can be free of bisphenol A, thus avoiding potentially harmful exposures of patients thereto. The fill valve can be provided with a “one-part” valve element, i.e. a valve element that consists of a single part, thus avoiding a need for manufacture of multiple valve element parts, a risk of failure in use associated with a multi-part valve element, and a need for multi-part assembly of the valve element. The fill valve can eliminate the need for a spring and a rigid valve housing. The fill valve can be used and accessed not only by trained healthcare professionals, but also by patients and caregivers. The fill valve can be manufactured easily and inexpensively. The fill valve can be used with a variety of medical devices, e.g. a variety of enteral feeding buttons, as well as other medical devices. The fill valve can be resistant to clogging and amenable to cleaning. The fill valve can have a valve element and valve housing, with the valve element being adhered securely to the valve housing. The fill valve can provide a straight flow path. The fill valve can withstand large back pressures.

Considering features of the fill valve for mediating two-way fluid communication between a medical instrument and a medical device in more detail, FIG. 1 provides a schematic illustration of an example fill valve 100.

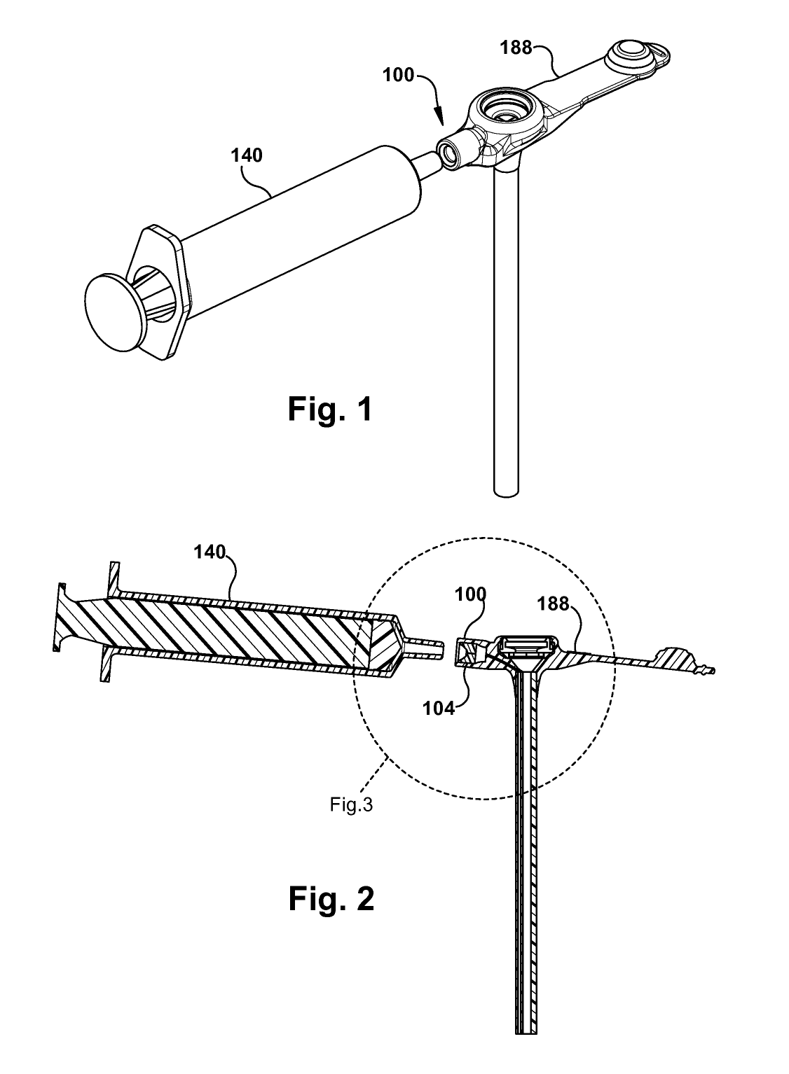

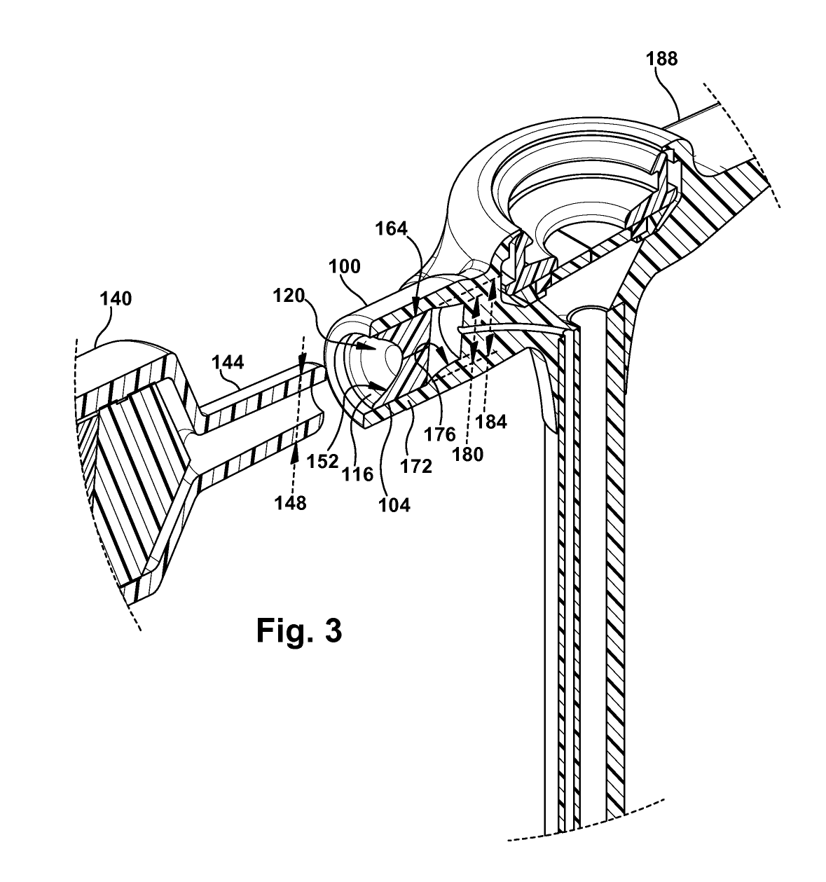

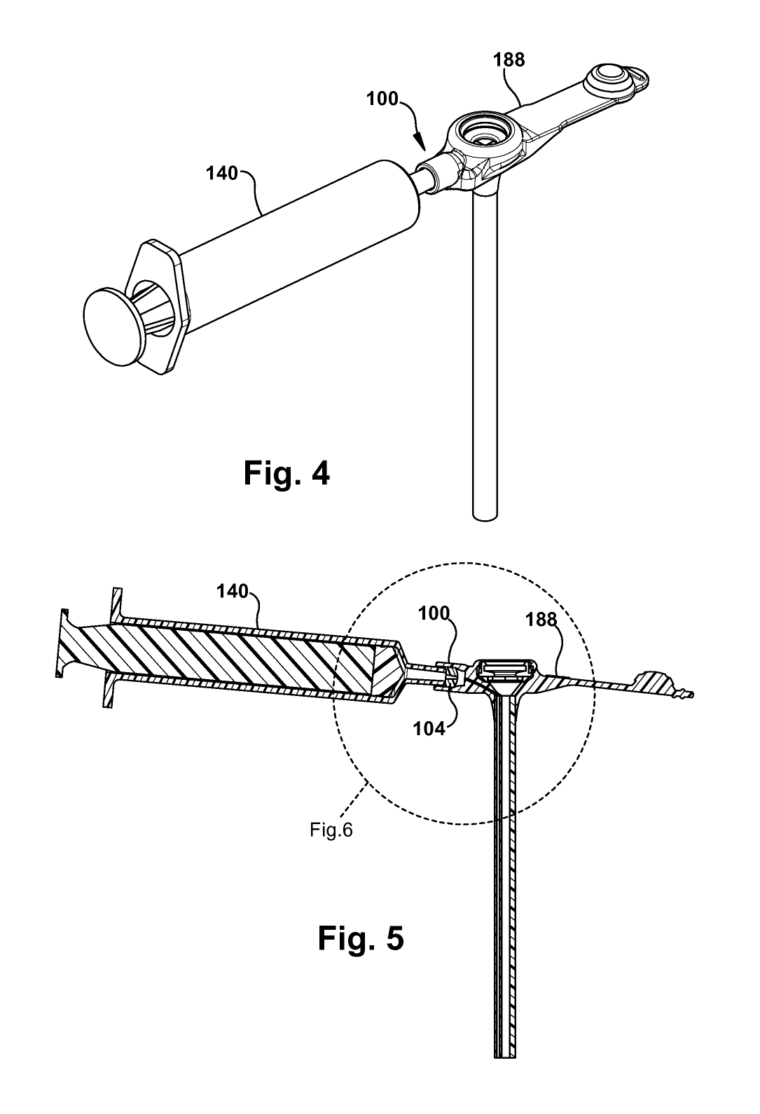

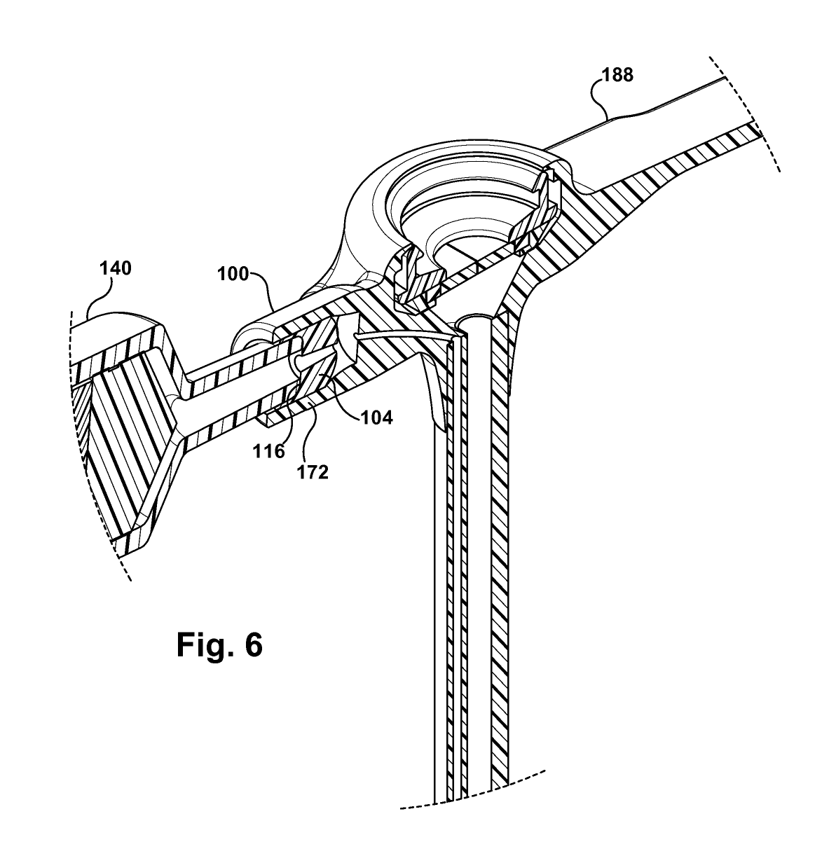

As shown in FIG. 2 and FIG. 3, the fill valve 100 includes a valve element 104. As shown in FIG. 4, FIG. 5, and FIG. 6, the fill valve 100 can mediate two-way fluid communication between a medical instrument and a medical device based on mediating flow of fluid, e.g. gas or liquid, through the valve element 104, based on the structure and material properties of the valve element 104, as follows.

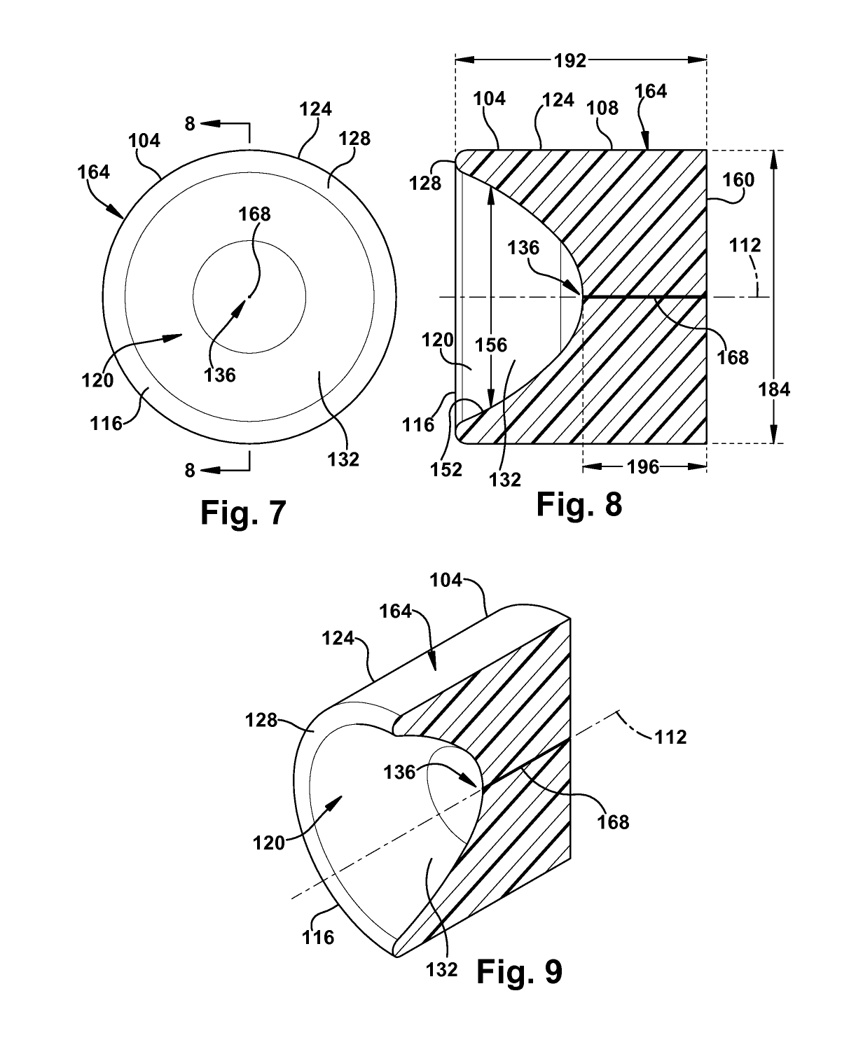

As shown in FIG. 7, FIG. 8, and FIG. 9, the valve element 104 includes a valve element body 108 extending along a major axis 112 of the valve element 104. As shown in FIG. 8 and FIG. 9, the valve element body 108 can have a shape that is, for example, substantially cylindrical, i.e. a shape that is cylindrical or essentially so, with a rounded contour defined by parallel lines, along all or most of the length of the valve element body 108. Alternatively, the valve element body 108 can have a shape that is substantially prismatic, i.e. a shape that is prismatic or essentially so, with a planar contour defined by parallel lines, again along all or most of the length of the valve element body 108, or substantially conical, i.e. a shape that is conical or essentially so, with a rounded contour defined by converging lines, again along all or most of the length of the valve element body 108, among other shapes. Also alternatively, the valve element body 108 can have a shape that is, for example, a curved, spiral, or irregular variant of a shape that is substantially cylindrical, substantially prismatic, or substantially conical, or combinations thereof. Accordingly the major axis 112 can be, for example, linear, curved, spiral, or irregular, among other types.

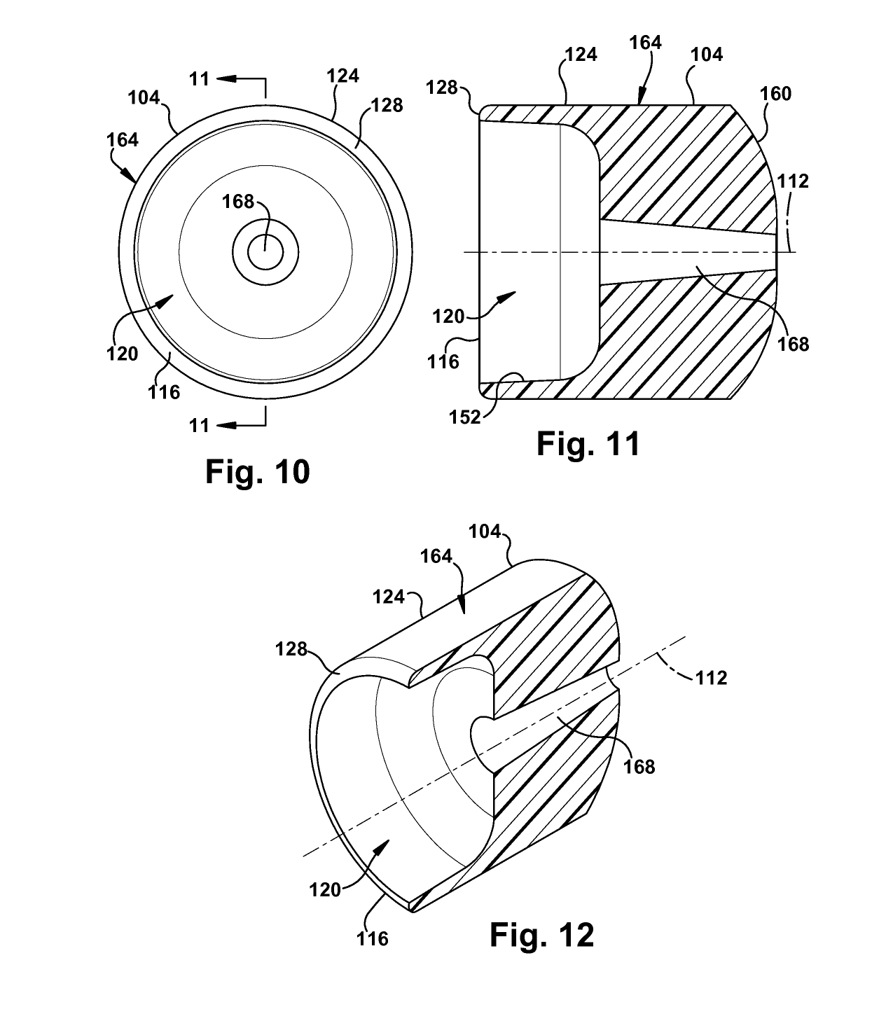

As shown in FIG. 6, FIG. 10, FIG. 11, and FIG. 12, the valve element 104 also includes a valve element first end 116 including a recessed internal surface 120 configured to provide a sealing contact engagement with a medical instrument 140. As shown in FIG. 7, FIG. 8, and FIG. 9, the recessed internal surface 120 can correspond, for example, to an internal surface of wall 124 of the valve element 104, and can extend, for example, from an edge 128 of the valve element 104. The recessed internal surface 120 can be, for example, contoured, angled, or flat, among other forms. The edge 128 can be, for example, rounded or contoured, among other forms. The recessed internal surface 120 can define a concave depression 132 receding along the major axis 112. The recessed internal surface 120 can include a nadir surface 136 that can be centered with respect to the major axis 112. The medical instrument 140 with which the recessed internal surface 120 of the valve element first end 116 is configured to provide the sealing contact engagement can be, for example, a syringe, a cannula, or a tube, among other medical instruments. The syringe can be a Luer-slip type syringe, a Luer-lock type syringe, an oral-tip type syringe, or an enteral-tip type syringe, among others.

With reference to FIG. 3, FIG. 6, FIG. 8, and FIG. 11, the sealing engagement can be based, for example, on the medical instrument 140 having a tip 144 having a tip external diameter 148, the recessed internal surface 120 having a contact surface 152 having a contact surface diameter 156, and the tip external diameter 148 being greater than the contact surface diameter 156, such that insertion of the tip 144 into the valve element 104 to or beyond the contact surface 152 results in compression contact between the recessed internal surface 120 and the tip 144, thus deterring fluid from leaking between the contact surface 152 and the tip 144, and thereby providing the sealing engagement. In accordance with this example, the tip 144 can be inserted into the valve element 104 readily and still provide the sealing engagement. Moreover, as noted above, the recessed internal surface 120 can correspond, for example, to an internal surface of wall 124 of the valve element 104, and can extend, for example, from an edge 128 of the valve element 104. In addition, as noted above, the edge 128 can be, for example, rounded or contoured, among other forms. In accordance with this example too, the tip 144 can be inserted into the valve element 104 readily and still provide the sealing engagement.

As shown in FIG. 8 and FIG. 11, the valve element 104 also includes a valve element second end 160 opposite the valve element first end 116. The valve element second end 160 can be, for example, flat, contoured, or angled, among other forms.

As shown in FIG. 7, FIG. 8, and FIG. 9, the valve element 104 also includes a valve element body external surface 164. As will be appreciated, the valve element shape is determined by the valve element body external surface 164.

The valve element 104 is resilient and deformable. Accordingly, the valve element 104 may be transformed from an original form or position to another upon application of a force thereto, and may return to the original form or position once the force is no longer being applied. The valve element 104 can be made, for example, of an elastomeric material selected from the group consisting of silicone, polyurethane, styrene ethylene butylene styrene block copolymer, polyvinylchloride, and combinations thereof, among other materials. The resilience and deformability of the valve element 104 promotes the sealing contact engagement between the recessed internal surface 120 and the medical instrument 140, e.g. between the recessed internal surface 120 and a Luer-slip type syringe, a Luer-lock type syringe, an oral-tip type syringe, and an enteral-tip type syringe, among others.

As shown in FIG. 7, FIG. 8, and FIG. 9, the valve element 104 also has a hole 168. The hole 168 extends along the major axis 112, from the valve element first end 116, to the valve element second end 160, through the valve element body 108. The hole 168 can be, for example a pin hole. The hole 168 also is substantially cylindrically symmetrical with respect to the major axis 112, i.e. a hole that is cylindrically symmetrical or essentially so, with a round form in cross section with respect to the major axis 112. In some examples, the hole 168 can close up, reversibly, in the absence of perturbation of the valve element 104, e.g. when the valve element 104 is not being used, such that fluid cannot pass through the hole 168. In other examples, the hole 168 can be open in the absence of perturbation of the valve element 104. Also, in some examples, the valve element has only one hole 168. In other examples, the valve element 104 has a plurality of holes 168, e.g. two, three, four, or more holes 168. Because the hole 168 is substantially cylindrically symmetrical with respect to the major axis 112, the hole 168 is not a slit, which would tend to have a linear or rectangular form in cross section with respect to the major axis 112.

As noted, the hole 168 extends along the major axis 112, from the valve element first end 116, to the valve element second end 160, through the valve element body 108. Also as noted, the recessed internal surface 120 can define a concave depression 132 receding along the major axis 112, and the recessed internal surface 120 can include a nadir surface 136 that can be centered with respect to the major axis 112. Thus, for example, the hole 168 can extend from the recessed internal surface 120 of the valve element first end 116, to the valve element second end 160, through the valve element body 108. Also for example, the hole 168 can extend from the concave depression 132 defined by the recessed internal surface 120 of the valve element first end 116, to the valve element second end 160, through the valve element body 108. Also for example, the hole 168 can extend from the nadir surface 136 of the recessed internal surface 120, to the valve element second end 160, through the valve element body 108. Also for example, the hole 168 can be centered with respect to the valve element 104, such that the hole 168 is aligned with the major axis 112. Thus, for example, the hole 168 can be centered with respect to the valve element 104, such that the hole 168 extends from the nadir surface 136 of the recessed internal surface 120 and both the hole 168 and the nadir surface 136 are aligned with the major axis 112.

The valve element 104 can be made, for example, as a single molded part. For example, in accordance with the disclosure above, the valve element 104 can be molded from an elastomeric material, e.g. silicone, to be, e.g. substantially cylindrical, including a valve element body 108, a valve element first end 116, e.g. including a recessed internal surface 120 defining a concave depression 132 and including a nadir surface 136, all centered with respect to a major axis 112, and also including a valve element second end 160, that is, e.g. flat, and a valve element body external surface 164, as described above. After molding, the elastomeric material of the valve element 104 can be cured, e.g. completely. Then a hole 168 can be formed through the valve element 104, as described above, e.g. by puncturing the valve element 104 with a thin wire or a sharp needle. The hole 168 can be formed without removing any material from the valve element 104, e.g. any portion of the valve element 104 that may be displaced by a sharp needle during the puncturing can remain part of the valve element 104. Curing the elastomeric material of the valve element 104 completely can ensure that the integrity of the hole 168 is maintained over time, e.g. by preventing the hole 168 from sealing shut permanently. As will be appreciated, the valve element 104 and the hole 168 also can be made and/or formed by other approaches.

As shown in FIG. 3 and FIG. 6, the fill valve 100 also includes a valve housing 172. Like the valve element 104, the valve housing 172 can be made, for example, of an elastomeric material selected from the group consisting of silicone, polyurethane, styrene ethylene butylene styrene block copolymer, polyvinylchloride, and combinations thereof, among other materials. In some examples, the valve housing 172 is made from the same elastomeric material as the valve element 104. In some examples, the valve housing 172 is made from a different elastomeric material than the valve element 104.

With reference to FIG. 3, the valve housing 172 is configured to hold the valve element 104 in place by a circumferential compression fit with the valve element body external surface 164. The circumferential compression fit can be based, for example, on a complementary fit between a valve housing internal surface 176 and the valve element body external surface 164, and thus between the valve housing 172 and the valve element 104. For example, as shown in FIG. 3, the valve element 104 can have a valve element external diameter 180, the valve housing 172 can have a valve housing internal diameter 184, and the valve element external diameter 180 can be greater than the valve housing internal diameter 184. In accordance with this example, based on the valve element 104 being resilient and deformable, the valve element 104 may be positioned within the valve housing 172 despite the valve element external diameter 180 being greater than the valve housing internal diameter 184. When so positioned, the valve housing 172 thereby holds the valve element 104 in place by a circumferential compression fit with the valve element body external surface 164. Moreover, when so positioned, the valve housing 172 deters leakage of fluid around the valve element 104.

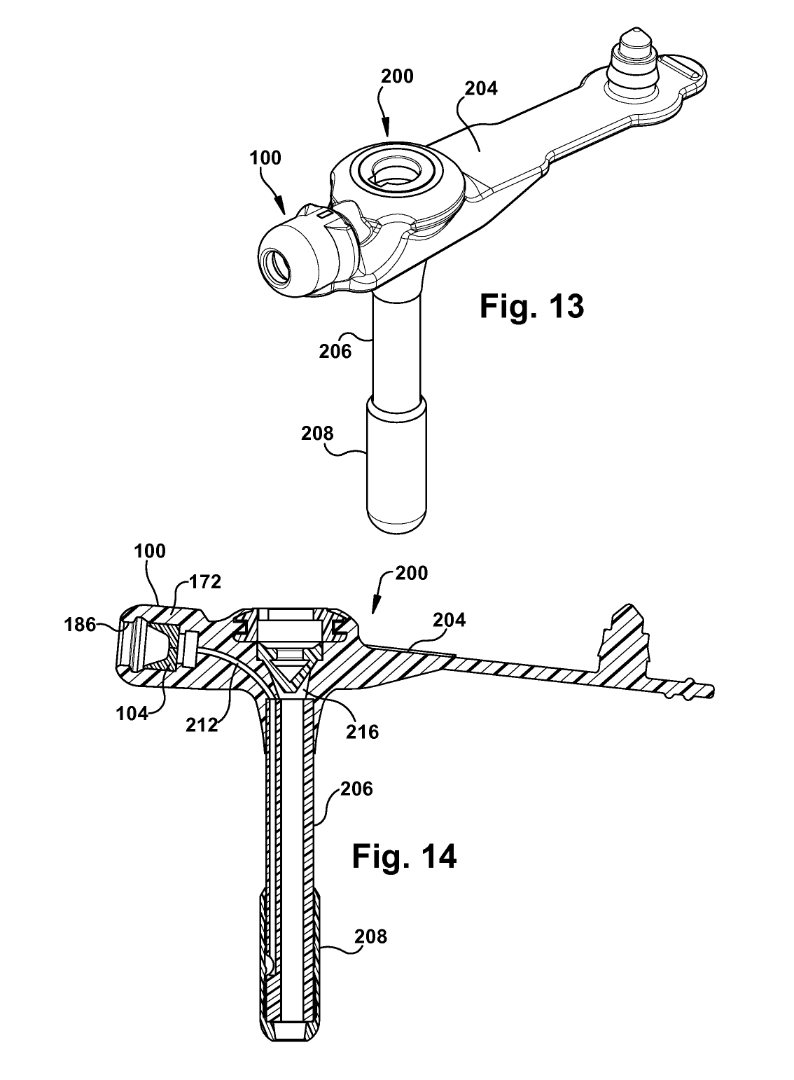

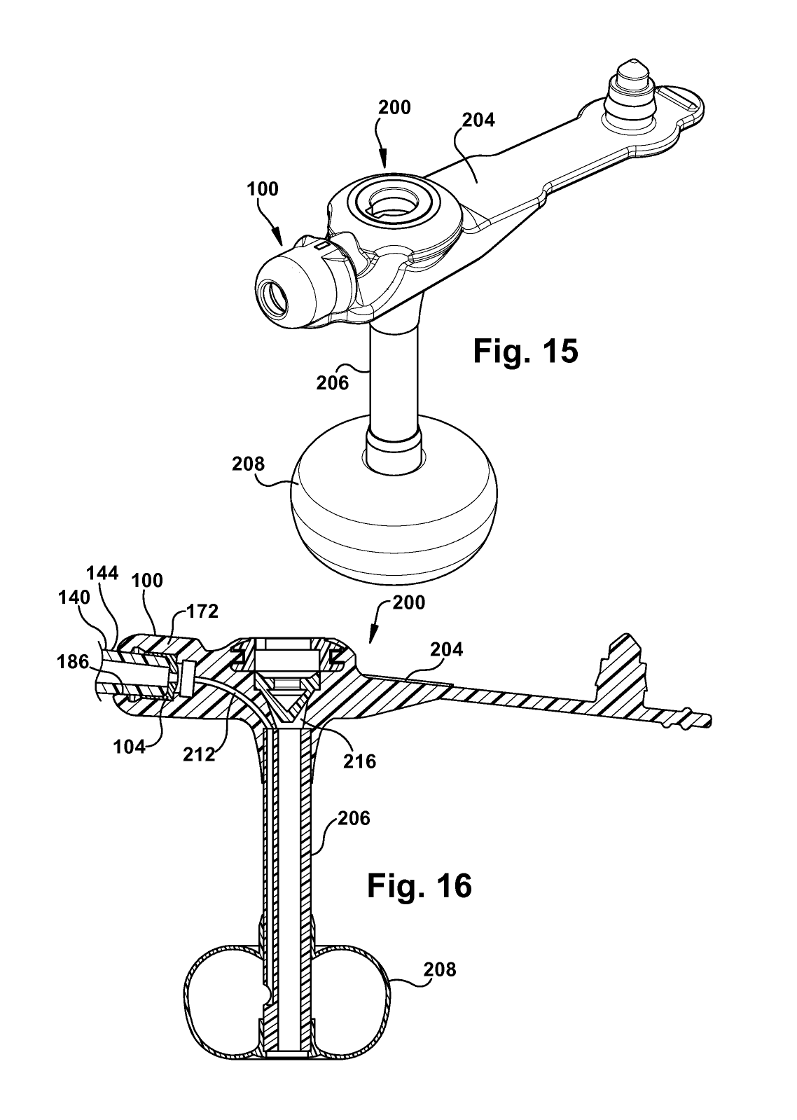

With reference to FIG. 13, FIG. 14, FIG. 15, and FIG. 16, in some embodiments, the valve housing 172 includes an inner gripping portion 186, such as an inner protruding ring or inner protruding tab, on the valve housing internal surface 176. As shown in FIG. 14, in some examples the inner gripping portion 186 can be integral to the valve housing 172, although in other examples the inner gripping portion 186 can be added to the valve housing 172 secondarily to manufacture of the valve housing 172. As shown in FIG. 16, the inner gripping portion 186 can be configured to provide a gripping pressure on the medical instrument 140, for example by decreasing the valve housing internal diameter 184, at a position along a path of the medical instrument 140 through the valve housing 172, in order to grip a tip 144 of the medical instrument 140 upon insertion of the tip 144 into the valve element 104, toward holding the tip 144 in place therein. For example, with reference to FIG. 3, FIG. 14, and FIG. 16, the inner gripping portion 186 can decrease the valve housing internal diameter 184, at a position along the path of the medical instrument 140 through the valve housing 172, to a diameter smaller than a tip external diameter 148 of the tip 144 of the medical instrument 140, such that as the tip 144 of the medical instrument 140 is being advanced toward and/or inserted in the valve element 104, the tip 144 also contacts the inner gripping portion 186, displacing the inner gripping portion 186, thereby causing the valve housing 172 to exert gripping pressure on the tip 144 and hold the tip 144 in place. The fill valve 100 may otherwise tend to push the tip 144 of the medical instrument 140 therefrom if the medical instrument 104 is not being pushed toward the fill valve 100, due to the resilience of the valve element 104 of the fill valve 100. In other embodiments, the valve housing 172 does not include such an inner gripping portion 186.

In some embodiments, the valve element 104 is centered with respect to the valve housing 172, such that the valve element 104 and the valve housing 172 are aligned with respect to the major axis 112. In other embodiments, the valve element 104 is not so centered.

As noted, the valve housing 172 is configured to hold the valve element 104 in place by a circumferential compression fit with the valve element body external surface 164. The valve housing 172 can further hold the valve element 104 in place based on adhesion of the valve element 104 to the valve housing 172. Thus, for example, in some embodiments, along with the circumferential compression fit, the valve element 104 is adhered to the valve housing 172, e.g. by use of an adhesive, such as a two-part silicone adhesive, for example to ensure that the valve element 104 stays in place during extended use and when subjected to relatively high back pressure, though in other embodiments, the valve element 104 is not adhered to the valve housing 172, e.g. no adhesive is used, for example to ensure that the valve element 104 can be replaced readily as desired or needed. As will be apparent from the discussion above, the valve element 104 can be positioned within, and adhered to, the valve housing 172, secondarily to manufacture of the valve housing 172.

As shown in FIG. 3, the valve housing 172 also is configured to connect to, or be an integral part of, a medical device 188. For example, the valve housing 172 can be configured to connect to the medical device 188, for example based on having a complementary fit, such that the valve housing 172 can be, or is, connected to the medical device 188, for example based on a press fit, a screw fit, adhesion, or soldering, among others. Thus, for example, the valve housing 172 can be connected to the medical device 188 secondarily to manufacture of the medical device 188. Alternatively, the valve housing 172 can be configured to be an integral part of the medical device 188, for example based on being formed as an integral part of the medical device 188 during manufacture of the medical device 188. Thus for example, the valve housing 172 can be formed during manufacture of, and as an integral part of, the medical device 188, for example as part of a molding process. Also alternatively, the valve housing 172 can be configured to be an integral part of the medical device 188, for example based on having a complementary fit, such that the valve housing 172 can be, or has been, added to the medical device 188 after manufacture of the medical device 188 such that the valve housing 172 would be, or is, an integral part of the medical device 188. Thus for example, the valve housing 172 can be made an integral part of the medical device 188 secondarily to manufacture of the medical device 188, e.g. by melding. As will be appreciated, the valve housing 172, as configured to connect to, or be an integral part of, a medical device 188, can be separate from the medical device 188, or can be connected thereto, or an integral part thereof, depending on whether or not the valve housing 172 has yet been connected thereto, or made an integral part thereof. The medical device 188 to which the valve housing 172 is configured to be connected to, or to be an integral part of, can be, for example, an enteral feeding device, an enteral feeding button, a gastrostomy balloon button, a gastrostomy-jejunostomy balloon button, a jejunostomy balloon button, a cecostomy balloon button, a balloon gastrostomy tube, an intravenous line, an arterial line, a dialysis line, a Foley catheter, or a chest tube, among others.

With reference to FIG. 3, FIG. 7, FIG. 8, and FIG. 9, the fill valve 100 is configured to, in the absence of the sealing contact engagement, maintain the hole 168 in a closed configuration, from the valve element first end 116, to the valve element second end 160, through the valve element body 108, based on the circumferential compression fit, thereby preventing two-way fluid communication through the hole 168. The fill valve 100 is so configured based on a combination of material properties, structure, and disposition thereof, namely that the valve element 104 is resilient and deformable, that the valve element 104 has the hole 168 that extends along the major axis 112, from the valve element first end 116, to the valve element second end 160, through the valve element body 108, and that is substantially cylindrically symmetrical with respect to the major axis 112, and that the valve housing 172 is configured to hold the valve element 104 in place by the circumferential compression fit. Because the fill valve 104 is resilient and deformable, when it is subjected to the circumferential compression fit, in the absence of the sealing contact engagement, the hole 168 closes, reversibly, from the valve element first end 116, to the valve element second end 160, through the valve element body 108. Moreover, as noted above, in some examples the hole 168 can close up, reversibly, in the absence of perturbation of the valve element 104, and thus can be closed even in the absence of the compression fit. The closed configuration of the hole 168 so maintained prevents fluid communication through the hole 168 in either direction while the hole 168 is in the closed configuration, thus preventing two-way fluid communication through the valve element 104 and thus the valve 100 while the hole 168 is in the closed configuration.

With reference to FIG. 6, FIG. 10, FIG. 11, and FIG. 12, the fill valve 100 also is configured to, in the presence of the sealing contact engagement, deform the hole 168, reversibly, to an open configuration, from the valve element first end 116, to the valve element second end 160, through the valve element body 108, despite the circumferential compression fit, thereby allowing two-way fluid communication through the hole 168. The fill valve 100 is so configured based again on a combination of material properties, structure, and disposition thereof, namely that the valve element 104 is resilient and deformable, that the valve element 104 has the hole 168 that extends along the major axis 112, from the valve element first end 116, to the valve element second end 160, through the valve element body 108, and that is substantially cylindrically symmetrical with respect to the major axis 112, and that the sealing engagement can be based, for example, on the medical instrument 140 having the tip 144 having the tip external diameter 148, the recessed internal surface 120 having the contact surface 152 having the contact surface diameter 156, and the tip external diameter 148 being greater than the contact surface diameter 156, such that insertion of the tip 144 into the valve element 104 to or beyond the contact surface 152 results in compression contact between the recessed internal surface 120 and the tip 144, thus providing the sealing engagement. Because the fill valve 104 is resilient and deformable, when it is subjected to the sealing contact engagement, despite the circumferential compression fit, the hole 168 deforms, e.g. by stretching, and thereby opens, reversibly, from the valve element first end 116, to the valve element second end 160, through the valve element body 108. For example, the hole 168 can deform to a truncated conical shaped opening in the open configuration, among other shaped openings. The open configuration of the hole 168 so maintained allows fluid communication through the hole 168 in both directions while the hole 168 is in the open configuration, thus allowing two-way fluid communication through the valve element 104 and thus the valve 100. Moreover, again because the fill valve 104 is resilient and deformable, when it is no longer subjected to the sealing contact engagement, e.g. based on withdrawal of the tip 144 of the medical instrument 140 from the valve element 104, the deformation of the hole 168 reverses, and the hole 168 reverts to the closed configuration, from the valve element first end 116, to the valve element second end 160, through the valve element body 108, based on the circumferential compression fit, thereby once again preventing two-way fluid communication through the hole 168. In addition, the hole 168 can be deformed to the open configuration, and reverted to the closed configuration, multiple times, during use of the fill valve 100, as needed.

Considering the fill valve 100 in more detail, in some examples, in the presence of the sealing contact engagement the valve housing 172 deforms, reversibly, adjacent to the valve element first end 116, near where the tip 144 of the medical instrument 140 has been inserted, although in other examples it does not. Such deformation of the valve housing 172 allows for compatibility of a single fill valve 100 with a variety of medical instruments 140 having tips 144 of a variety of sizes. For example, considering medical instruments 140 corresponding to syringes in particular, certain types of syringe tips are comparatively larger than others, e.g. tips of oral-tip type syringes and enteral-tip type syringes are comparatively larger than tips of Luer-slip type syringes and Luer-lock type syringes. Because the valve housing 172 can be so deformed, a single particular fill valve 100 can be compatible for use with a variety of syringes having tips of a variety of sizes, e.g. oral-tip type syringes, enteral-tip type syringes, Luer-slip type syringes, and Luer-lock type syringes.

Also, in some examples, in the presence of the sealing contact engagement, the valve element 104 bulges, reversibly, at the valve element second end 160, although in other examples it does not. Like deformation of the valve housing 172, such bulging of the valve element 104 allows for compatibility of a single fill valve 100 with a variety of medical instruments 140 having tips 144 of a variety of sizes.

As will be appreciated from the disclosure above, the fill valve 100 thus mediates two-way fluid communication between a medical instrument and a medical device, by preventing two-way fluid communication through the valve element 104 and thus the valve 100 in the absence of the sealing contact engagement between the recessed internal surface 120 and a medical instrument 140, based on the circumferential compression fit, while allowing two-way fluid communication through the valve element 104 and thus the valve 100 in the presence of the sealing contact engagement between the recessed internal surface 120 and the medical instrument 140, despite the circumferential compression fit.

With reference to FIG. 8, various dimensions of the fill valve 100, including of the valve element 104 and the valve housing 172 thereof, can contribute to the fill valve 100 being configured, in the absence of the sealing contact engagement, to maintain the hole 168 in a closed configuration, as described, and being configured, in the presence of the sealing contact engagement, to deform the hole 168, reversibly, to an open configuration, also as described. For example, in some embodiments the valve element 104 has a valve element length 192 along the major axis 112, wherein the valve element length 192 is, e.g. 0.4 mm to 20 mm, 1.0 mm to 10 mm, or 1.5 mm to 5 mm. Also for example, in some embodiments the valve element 104 has a valve element external diameter 180 corresponding to, e.g. 1 mm to 30 mm, 1.5 mm to 15 mm, or 2 mm to 8 mm. Also for example, in some embodiments the valve element 104 has a valve element length 192 along the major axis 112, and the valve element 104 has a valve element external diameter 180, wherein the valve element length 192 is less than the valve element external diameter 180, e.g. the ratio of the valve element length 192 to the valve element external diameter 180 is less than 1:1, less than 0.9:1, less than 0.8:1, less than 0.7:1, less than 0.6:1, or less than 0.5:1. Also for example, in some embodiments, the hole 168 has a hole length 196 along the major axis 112, wherein the hole length 196 is 0.3 mm to 15 mm, 0.4 mm to 5 mm or 0.5 to 2 mm. Also for example, in some embodiments, the valve element 104 has a valve element length 192 along the major axis 112, the hole 168 has a hole length 196 along the major axis 112, and the hole length 196 is less than the valve element length 192, e.g. the ratio of the hole length 196 to the valve element length 192 is less than 0.6:1, less than 0.5:1, less than 0.4:1, less than 0.3:1, or less than 0.2:1.

As noted above, the hole 168 is substantially cylindrically symmetrical with respect to the major axis 112, and thus the hole 168 is not a slit. Because the hole 168 is substantially cylindrically symmetrical with respect to the major axis 112, and thus is not a slit, application of pressure to the valve other than by the sealing contact engagement, e.g. asymmetrical lateral pressure at the valve element body external surface 164 or back pressure at the valve element second end 160 does not result in opening of the hole 168, in contrast to a slit.

An example enteral feeding button also is provided. One use for the fill valve 100 is in connection with enteral feeding buttons. This is because enteral feeding buttons often include use of a balloon to hold the button in place in a patient, the balloon is often filled via a fill valve that is external to the patient, and the various advantages noted above for the fill valve 100 particularly apply with respect to use with enteral feeding buttons. Moreover, the use of a soft material for both the fill valve 100 and the enteral feeding button provides a more comfortable and reliable enteral feeding button for patients.

Considering features of the enteral feeding button in more detail, FIG. 13 provides a schematic illustration of an example enteral feeding button 200.

As shown in FIG. 13, FIG. 14, FIG. 15, and FIG. 16, the enteral feeding button 200 includes an enteral feeding button body 204, an enteral feeding tube 206, and a balloon 208. The enteral feeding button body 204, the enteral feeding tube 206, and the balloon 208 are connected in series in fluid communication. As shown in FIG. 13 and FIG. 14, the balloon 208 can be deflated, such that the balloon 208 is in a deflated state. Also, as shown in FIG. 15 and FIG. 16, the balloon 208 can be inflated, such that the balloon 208 is in an inflated state.

As shown in FIG. 14 and FIG. 16, the enteral feeding button body 204 includes a channel 212 for inflating or deflating the balloon 208 through the enteral feeding tube 206. The enteral feeding button body 204 also includes a channel 216 for receiving a feeding solution. The enteral feeding button body 204 also includes a fill valve 100 for mediating two-way fluid communication between a medical instrument 140 and the channel 212 for inflating or deflating the balloon 208.

The fill valve 100 includes a valve element 104, as discussed above. Thus, as shown in FIG. 7, FIG. 8, and FIG. 9, the valve element 104 includes (i) a valve element body 108 extending along a major axis 112 of the valve element 104, (ii) a valve element first end 116 including a recessed internal surface 120 configured to provide a sealing contact engagement with the medical instrument 140, (iii) a valve element second end 160 opposite the valve element first end 116, and (iv) a valve element body external surface 164. The valve element 104 also is resilient and deformable. The valve element 104 also has a hole 168 that extends along the major axis 112, from the valve element first end 116, to the valve element second end 160, through the valve element body 108, and that is substantially cylindrically symmetrical with respect to the major axis 112.

The fill valve 100 also includes a valve housing 172, as discussed above. Thus, the valve housing 172 is configured to hold the valve element 104 in place by a circumferential compression fit with the valve element body external surface 164. In this case, the valve housing 172 is an integral part of the enteral feeding button body 204.

The fill valve 100 is configured as discussed above. Thus, the fill valve 100 is configured to, in the absence of the sealing contact engagement, maintain the hole 168 in a closed configuration, from the valve element first end 116, to the valve element second end 160, through the valve element body 108, based on the circumferential compression fit, thereby preventing two-way fluid communication through the hole 168, as discussed above. The fill valve 100 also is configured to, in the presence of the sealing contact engagement, deform the hole 168, reversibly, to an open configuration, from the valve element first end 116, to the valve element second end 160, through the valve element body 108, despite the circumferential compression fit, thereby allowing two-way fluid communication through the hole 168, also as discussed above.

As discussed above, the valve element 104 can be made, for example, of an elastomeric material selected from the group consisting of silicone, polyurethane, styrene ethylene butylene styrene block copolymer, polyvinylchloride, and combinations thereof, among other materials. Moreover, the valve housing 172 can be made, for example, of an elastomeric material selected from the group consisting of silicone, polyurethane, styrene ethylene butylene styrene block copolymer, polyvinylchloride, and combinations thereof, among other materials.

Also as discussed above, the valve element 104 can be substantially cylindrical. Furthermore, (i) the valve element 104 can have a valve element external diameter 180, (ii) the valve housing 172 can have a valve housing internal diameter 184, and (iii) the valve element external diameter 180 can be greater than the valve housing internal diameter 184.

Also as discussed above, the hole 168 can be centered with respect to the valve element 104. Moreover, the valve element 104 can centered with respect to the valve housing 172.

Also as discussed above, the hole 168 can deform to a truncated conical shaped opening in the open configuration. Moreover, the recessed internal surface 120 can define a concave depression 132 receding along the major axis 112, and the hole 168 can extend from the concave depression 132, to the valve element second end 160, through the valve element body 108.

Also as discussed above, the valve element 104 can have a valve element length 192 along the major axis 112, the hole 168 can have a hole length 196 along the major axis 112, and the hole length 196 can be less than the valve element length 192.

Also as discussed above, the medical instrument 140 with which the recessed internal surface 120 of the valve element first end 116 is configured to provide the sealing contact engagement can be a syringe. The enteral feeding button 200 can be, for example, a gastrostomy balloon button, a gastrostomy-jejunostomy balloon button, or a jejunostomy balloon button.

An example valve element also is provided. Considering features of the valve element in more detail, FIG. 7, FIG. 8, and FIG. 9 provide a schematic illustration of an example valve element 104.

The valve element 104 can be as discussed above. Thus, the valve element 104 includes (i) a valve element body 108 extending along a major axis 112 of the valve element 104, (ii) a valve element first end 116 including a recessed internal surface 120 configured to provide a sealing contact engagement with a medical instrument 140, (iii) a valve element second end 160 opposite the valve element first end 116, and (iv) a valve element body external surface 164. The valve element 104 also is resilient and deformable. The valve element 104 also has a hole 168 that extends along the major axis 112, from the valve element first end 116, to the valve element second end 160, through the valve element body 108, and that is substantially cylindrically symmetrical with respect to the major axis 112.

In accordance with the discussion above, the valve element 104 is configured to, in the absence of the sealing contact engagement, maintain the hole 168 in a closed configuration, from the valve element first end 116, to the valve element second end 160, through the valve element body 108, based on a circumferential compression fit between a valve housing 172 and the valve element body external surface 164, thereby preventing two-way fluid communication through the hole 168. The valve element 104 also is configured to, in the presence of the sealing contact engagement, deform the hole 168, reversibly, to an open configuration, from the valve element first end 116, to the valve element second end 160, through the valve element body 108, despite the circumferential compression fit, thereby allowing two-way fluid communication through the hole 168.

As discussed above, the valve element 104 can be made, for example, of an elastomeric material selected from the group consisting of silicone, polyurethane, styrene ethylene butylene styrene block copolymer, polyvinylchloride, and combinations thereof, among other materials. Moreover, the valve element 104 can be substantially cylindrical. In addition, the hole 168 can be centered with respect to the valve element 104. Furthermore, the hole 168 can deform to a truncated conical shaped opening in the open configuration. Further still, the recessed internal surface 120 can define a concave depression 132 receding along the major axis 112, and the hole 168 can extend from the concave depression 132, to the valve element second end 160, through the valve element body 108.

Also as discussed above, the valve element 104 can have a valve element length 192 along the major axis 112, the hole 168 can have a hole length 196 along the major axis 112, and the hole length 196 can be less than the valve element length 192.

Also as discussed above, the medical instrument 140 with which the recessed internal surface 120 of the valve element first end 116 is configured to provide the sealing contact engagement can be a syringe.

### Examples

As noted above, FIG. 13, FIG. 14, FIG. 15, and FIG. 16 show various schematic views of an example enteral feeding button. More specifically, the figures show a gastrostomy balloon button that includes an exemplary fill valve as a component of the gastrostomy balloon button body thereof. The fill valve is used as a balloon fill/check valve in order to inflate a balloon connected to the gastrostomy balloon button via the feeding tube of the gastrostomy balloon button, and keep the balloon inflated, to hold the feeding tube in the stomach of a patient. The fill valve also is used as a balloon fill/check valve in order to deflate the balloon, allowing removal of the feeding tube from the stomach of the patient. The fill valve is positioned on a side of the gastrostomy button body so that the fill valve does not interfere with a feeding orifice, which is positioned on the top of the gastrostomy button body. The gastrostomy button body includes a valve element and a valve housing in which the valve element is positioned.

In this example, the valve element is made in the form of a small cylinder from a soft, elastomeric material, such as low durometer silicone. One end of the cylinder has a concave shape. The concave shape serves as a receptacle for Luer syringes, e.g. Luer-slip type syringes or Luer-lock type syringes, based on the concave shape complementing the shape of a Luer syringe tip. The opposite end of the cylinder is a flat surface, perpendicular to the major axis of the cylinder. The Luer syringe tip can open the fill valve by interacting with the concave surface of the valve element and causing the valve element, and the valve housing in which the valve element is positioned, to stretch. Specifically, there is a small hole along the main cylinder axis. This hole, established after the molding process, is created using a thin wire to pierce the valve element along the main cylinder axis. No material is actually removed during the piercing process. The hole of the valve element is in a closed configuration when a syringe tip is not inserted in the valve element, and is in an open configuration when a syringe tip is inserted in the valve element. The valve element has a valve element length of 3.2 mm along the major axis, a valve element external diameter of 4.5 mm, and a hole length of 0.84 mm along the major axis, extending from the lowest part of the concave shaped end of the valve element, through the valve element, to the flat surfaced opposite end of the valve element, centered with respect to the major axis.

In this example, the valve housing is an integral part of the gastrostomy button body and also is made of an elastomeric material, such as low durometer silicone. The valve housing includes an opening, which is tubular, and a valve housing internal surface. As noted above, the valve element is positioned within the valve housing. In this case, the valve element has an outer diameter and the opening in the valve housing has an inner diameter, wherein the outer diameter of the valve element is greater than the inner diameter of the opening of the valve housing, i.e. the valve element is compressed to some extent as it is pushed into the valve housing, and the valve housing is displaced to some extent to accommodate the valve element therein. The valve housing also includes an inner gripping portion, in this case an inner protruding ring, on the valve housing internal surface, that is configured to provide a gripping pressure on a syringe tip, in order to grip the tip upon insertion of the tip into the valve element, toward holding the tip in place therein. The inner gripping portion is integral to the valve housing and thus to the gastrostomy button body. The valve element is adhered securely to the valve housing, in this case by use of a two-part silicone adhesive, to withstand backpressure.

The balloon is connected to the fill valve via a channel for inflating or deflating the balloon.

The fill valve of the gastrostomy balloon button can be operated as follows. In order to inflate or deflate the balloon, a syringe is aligned with the opening of the valve housing and then inserted into the concave shaped end of the valve element therein so that the leading end of the syringe, i.e. the syringe tip, presses against a recessed internal surface of the valve element, thus deforming the hole of the valve element to an open configuration. A plunger of the syringe can then be pressed forward in order to push liquid and/or gas from an internal chamber of the syringe, through the hole of the valve element, then through the channel for inflating or deflating the balloon, extending from the fill valve, through the gastrostomy button body and the feeding tube, to an internal chamber of the balloon. Alternatively, the plunger of the syringe can be pulled backward in order to draw liquid and/or gas, from the internal chamber of the balloon, through the channel for inflating or deflating the balloon, then through the hole of the valve element, to the internal chamber of the syringe. As discussed above, the hole can deform to a truncated conical shaped opening in the open configuration. Liquid or gas can enter or exit through this opening when the syringe is so inserted and applied. This can be demonstrated, for example, by so inserting the syringe into the valve element of a fill valve of a gastrostomy balloon button of which the balloon thereof already contains a liquid, e.g. water. Upon doing so, backpressure from the balloon causes the liquid to flow from the balloon, through the channel, through the hole of the valve element, into the internal chamber of the syringe. In contrast, when no syringe is so inserted, the liquid does not flow. When the syringe is removed, the deformation of the hole reverses, the hole reverts to the closed configuration, and liquid and gas can no longer pass through the hole. Thus, for example, once a syringe has been used to inflate the balloon as described above, and then is removed, the balloon remains inflated, thus holding the feeding tube in the stomach of a patient. Also for example, once a syringe has been used to deflate the balloon as described above, and then is removed, the balloon remains deflated, allowing removal of the feeding tube from the stomach of the patient.

While various features are presented above, it should be understood that the features may be used singly or in any combination thereof. It will be apparent to those skilled in the art that various modifications and variations can be made without departing from the spirit and scope of the claimed invention.

What is claimed is:

1. A fill valve for mediating two-way fluid communication between a medical instrument and a medical device, the fill valve comprising:
(a) a valve element comprising (i) a valve element body extending along a major axis of the valve element, (ii) a valve element first end comprising a recessed internal surface configured to provide a sealing contact engagement with a medical instrument, (iii) a valve element second end opposite the valve element first end, and (iv) a valve element body external surface, wherein the valve element is resilient and deformable and has a hole that extends along the major axis, from the valve element first end, to the valve element second end, through the valve element body, and that is substantially cylindrically symmetrical with respect to the major axis; and
(b) a valve housing configured to hold the valve element in place by a circumferential compression fit with the valve element body external surface and to connect to, or be an integral part of, a medical device,
wherein:
(1) the fill valve is configured to, in the absence of the sealing contact engagement, maintain the hole in a closed configuration, from the valve element first end, to the valve element second end, through the valve element body, based on the circumferential compression fit, thereby preventing two-way fluid communication through the hole; and
(2) the fill valve is configured to, in the presence of the sealing contact engagement, deform the hole, reversibly, to an open configuration, from the valve element first end, to the valve element second end, through the valve element body, despite the circumferential compression fit, thereby allowing two-way fluid communication through the hole.

2. The fill valve of claim 1, wherein the valve element is made of an elastomeric material selected from the group consisting of silicone, polyurethane, styrene ethylene butylene styrene block copolymer, polyvinylchloride, and combinations thereof.

3. The fill valve of claim 1, wherein the valve housing is made of an elastomeric material selected from the group consisting of silicone, polyurethane, styrene ethylene butylene styrene block copolymer, polyvinylchloride, and combinations thereof.

4. The fill valve of claim 1, wherein the valve element is substantially cylindrical.

5. The fill valve of claim 1, wherein: (i) the valve element has a valve element external diameter, (ii) the valve housing has a valve housing internal diameter, and (iii) the valve element external diameter is greater than the valve housing internal diameter.

6. The fill valve of claim 1, wherein the hole is centered with respect to the valve element.

7. The fill valve of claim 1, wherein the valve element is centered with respect to the valve housing.

8. The fill valve of claim 1, wherein the hole deforms to a truncated conical shaped opening in the open configuration.

9. The fill valve of claim 1, wherein the recessed internal surface defines a concave depression receding along the major axis, and the hole extends from the concave depression, to the valve element second end, through the valve element body.

10. The fill valve of claim 1, wherein the valve element has a valve element length along the major axis, the hole has a hole length along the major axis, and the hole length is less than the valve element length.

11. The fill valve of claim 1, wherein the valve housing includes an inner gripping portion.

12. The fill valve of claim 1, wherein the medical instrument with which the recessed internal surface of the valve element first end is configured to provide the sealing contact engagement is a syringe, a cannula, or a tube.

13. The fill valve of claim 1, herein the medical device to which the valve housing is configured to be connected to, or to be an integral part of, is an enteral feeding device, an enteral feeding button, a gastrostomy balloon button, a gastrostomy-jejunostomy balloon button, a jejunostomy balloon button, a cecostomy balloon button, a balloon gastrostomy tube, an intravenous line, an arterial line, a dialysis line, a Foley catheter, or a chest tube.

14. An enteral feeding button comprising an enteral feeding button body, an enteral feeding tube, and a balloon, wherein:
the enteral feeding button body, the enteral feeding tube, and the balloon are connected in series in fluid communication;
the enteral feeding button body comprises a channel for inflating or deflating the balloon, a channel for receiving a feeding solution, and a fill valve for mediating two-way fluid communication between a medical instrument and the channel for inflating or deflating the balloon; and
the fill valve comprises:
(a) a valve element comprising (i) a valve element body extending along a major axis of the valve element, (ii) a valve element first end comprising a recessed internal surface configured to provide a sealing contact engagement with the medical instrument, (iii) a valve element second end opposite the valve element first end, and (iv) a valve element body external surface, wherein the valve element is resilient and deformable and has a hole that extends along the major axis, from the valve element first end, to the valve element second end, through the valve element body, and that is substantially cylindrically symmetrical with respect to the major axis; and
(b) a valve housing configured to hold the valve element in place by a circumferential compression fit with the valve element body external surface and that is an integral part of the enteral feeding button body,
wherein:
(1) the fill valve is configured to, in the absence of the sealing contact engagement, maintain the hole in a closed configuration, from the valve element first end, to the valve element second end, through the valve element body, based on the circumferential compression fit, thereby preventing two-way fluid communication through the hole; and
(2) the fill valve is configured to, in the presence of the sealing contact engagement, deform the hole, reversibly, to an open configuration, from the valve element first end, to the valve element second end, through the valve element body, despite the circumferential compression fit, thereby allowing two-way fluid communication through the hole.

15. The enteral feeding button of claim 14, wherein the valve element is made of an elastomeric material selected from the group consisting of silicone, polyurethane, styrene ethylene butylene styrene block copolymer, polyvinylchloride, and combinations thereof.

16. The enteral feeding button of claim 14, wherein the valve housing is made of an elastomeric material selected from the group consisting of silicone, polyurethane, styrene ethylene butylene styrene block copolymer, polyvinylchloride, and combinations thereof.

17. The enteral feeding button of claim 14, wherein the valve element is substantially cylindrical.

18. The enteral feeding button of claim 14, wherein: (i) the valve element has a valve element external diameter, (ii) the valve housing has a valve housing internal diameter, and (iii) the valve element external diameter is greater than the valve housing internal diameter.

19. The enteral feeding button of claim 14, wherein the hole is centered with respect to the valve element.

20. The enteral feeding button of claim 14, wherein the valve element is centered with respect to the valve housing.

21. The enteral feeding button of claim 14, wherein the hole deforms to a truncated conical shaped opening in the open configuration.

22. The enteral feeding button of claim 14, wherein the recessed internal surface defines a concave depression receding along the major axis, and the hole extends from the concave depression, to the valve element second end, through the valve element body.

23. The enteral feeding button of claim 14, wherein the valve element has a valve element length along the major axis, the hole has a hole length along the major axis, and the hole length is less than the valve element length.

24. The enteral feeding button of claim 14, wherein the valve housing includes an inner gripping portion.

25. The enteral feeding button of claim 14, wherein the medical instrument with which the recessed internal surface of the valve element first end is configured to provide the sealing contact engagement is a syringe, a cannula, or a tube.

26. A valve element comprising:
(i) a valve element body extending along a major axis of the valve element,
(ii) a valve element first end comprising a recessed internal surface configured to provide a sealing contact engagement with a medical instrument,
(iii) a valve element second end opposite the valve element first end, and
(iv) a valve element body external surface,
wherein the valve element is resilient and deformable and has a hole that extends along the major axis, from the valve element first end, to the valve element second end, through the valve element body, and that is substantially cylindrically symmetrical with respect to the major axis; and
wherein:
(1) the valve element is configured to, in the absence of the sealing contact engagement, maintain the hole in a closed configuration, from the valve element first end, to the valve element second end, through the valve element body, based on a circumferential compression fit between a valve housing and the valve element body external surface, thereby preventing two-way fluid communication through the hole; and
(2) the valve element is configured to, in the presence of the sealing contact engagement, deform the hole, reversibly, to an open configuration, from the valve element first end, to the valve element second end, through the valve element body, despite the circumferential compression fit, thereby allowing two-way fluid communication through the hole.
